(19) United States
(12) Patent Application Publication
(10) Pub. No.: US 2004/0115234 A1
(43) Pub. Date: Jun. 17, 2004

(54) Cosmetic composition

(76) Inventor: Joan T. Gewirtz, Stamford, CT (US)

Correspondence Address: Evelyn M. Sommer, 30th Floor, 825 Third Avenue, New York, NY, 10022, US

(21) Appl. No.: 10/670,056

(22) Filed: Sep. 24, 2003

Related U.S. Application Data
(60) Provisional application No. 60/412,942, filed on Sep. 24, 2002.

(51) Int. Cl. A61K 7/00

(52) U.S. Cl. 424/401

(57) ABSTRACT

Methods and compositions for increasing the thickness, length and for darkening eyelashes comprising a prostaglandin, preferably Latanoprost in the form of a cosmetic preparation suitable for application to the eyelids at the site where the eyelashes are present.

This application claims priority of Provisional Application Serial No. 60/412942 filed Sep. 24, 2002, which is incorporated herein in its entirety by reference.

### FIELD OF THE INVENTION

The invention relates to a method for stimulating the growth and coloration of human eyelashes, comprising the application to the eyelids at the site where the eyelashes are present, of a prostaglandin compound, in the form of its solution in an ophthalmologically acceptable carrier, or incorporated into a commercial mascara or eyelash cosmetic preparation.

More particularly, the invention relates to the method for stimulating the growth and improving the coloration (darkening) of human eyelashes comprising the topical application of a composition comprising Latanoprost in an ophthalmologically acceptable carrier or incorporated into the conventional mascara or eyelash cosmetic preparation to the eyelids at the site of the eyelashes. More particularly the invention relates to the thickening, lengthening and darkening of human eyelashes for cosmetic purposes using the said compositions and namely using them by topically applying the composition to the eyelids at the site of the eyelashes.

### BACKGROUND OF THE INVENTION

Attempts to improve the appearance of eyelashes by lengthening, thickening and coloring them have not been entirely successful with products currently available. The preparations which rely solely on coating the existing eyelashes in some instances thicken them but cannot increase their apparent length significantly. Those products that contain fiber filaments admixed into their formulations cannot be applied to give an evenly distributed groomed appearance, and rather leave the lashes looking uneven, matted and gummy. A preparation employing the “one-on-one technique”, wherein filaments are glued into place one at a time, presents the problem of frustratingly laborious, painstaking, time-consuming work for the user. False eyelashes, where the lashes are previously mounted on a strip and the strip is designed to be glued to the eyelid, are uncomfortable to wear and can be kept in place for only a few hours at a time. U.S. Pat. No. 4,135,527 teaches an improved two-component method that can be used to bind fibers, using an adhesive, to the natural eyelashes to extend the length of eyelashes. However, this method is also difficult to implement.

Certain therapeutic agents have been known to induce hair growth in extensive areas of the trunk, limbs and even occasionally on the face of humans. Hypertrichosis (extensive growth of hair) has been observed in patients taking diphenylhydantoin, which is an anticonvulsant drug used to control epileptic seizures, and streptomycin when administered to children suffering tuberculous meningitis. Another example is Minoxidil attributable to Upjohn. The chemical name for Minoxidil is 6-(1-piferidinyl)-2,4-pyrimidane-diamine 3-oxide (U.S. Pat. Nos. 3,382,247 and 3,644,363). Minoxidil compounds were originally prepared and sold for use as antihypertensives. It was observed that associated with the use of Minoxidil for this latter purpose, an increase in hair growth and thickness. This has been reported in U.S. Pat. Nos. 4,139,619 and 4,968,812. Today Minoxidil is also marketed under the trademark Rogaine® by Pfizer for the treatment of baldness on the scalp for men (alopecia androgenetica) and women and may be purchased in almost every drug store, super market, and cosmetic sales outlet for this latter purpose with no prescription being required. Another example is finasteride (Propecia®), marketed by Merck & Co. Finasteride was originally developed for benign prostatic hypertrophy, and was found to be effective in the treatment of alopecia androgenetica. This has been reported in U.S. Pat. No. 4,968,812.

Among the drugs introduced for lowering intraocular pressure is prostaglandin F2. Clinical studies have established that the isopropyl ester derivative of prostaglandin F2 PhXA34, lowers intraocular pressure with minimal side effects (Camras, et al, Ophthalmology 1989, 96:1329-1336). The studies suggest that this prostaglandin derivative acts by increasing the uveoscleral outflow, i.e., the unconventional aqueous drainage route (Crawford and Kaufman, Archives of Ophthalmology, 1987, 105: 1112-1116).

In 1972, The Upjohn Company obtained U.S. Pat. No. 3,657,327 for Prostin F2α, commonly known as Prostaglandin F2α, 7-[3,5-dihydroxy-2-(3-hydroxy-oct-1-enyl)-cyclopentyl]-hept-5 -enoic acid, having the structural formula,

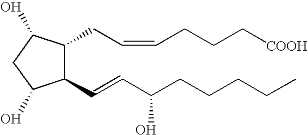

This compound functioned as a smooth muscle relaxant. U.S. Pat. No. 3,657,327 is incorporated herein in its entirety by reference thereto.

Subsequently Upjohn discovered that a prostaglandin F2α analogue, commonly known as Latanoprost and whose chemical name is isopropyl-(Z)-7[(1R, 2R, 3R, 5S)3,5-dihydroxy-2-[(3R)-3-hydroxy-5-phenylpentyl]cyclopentyl]-5-heptenoate, having a molecular formula of C₂₆H₄₀O₅, a molecular weight of 432.58, and a chemical structure,

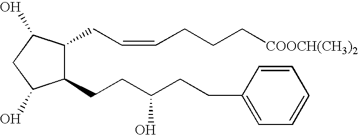

is a prostanoid selective FP receptor agonist which acts to reduce the intraocular pressure. Studies carried out in animals and men indicate that the main mechanism of action is increased uveoscleral outflow.

Latanoprost is marketed by Pharmacia & Upjohn (currently a part of Pfizer) under the trademark Xalatan for the reduction of elevated intraocular pressure in patients with glaucoma and ocular hypertension. Latanoprost is marketed in the form of a Latanoprost optical solution of 0.005 % (50 μg/ml), and is applied by dropper directly onto the eye. One drop contains approximately 1.5 μg of Latanoprost. It is absorbed through the cornea where the isopropyl ester pro-drug is hydrolyzed to the acid form to become biologically active.

The use of prostaglandins and their derivatives for lowering intraocular pressure is well documented in the literature, e.g. U.S. Pat. No. 6,262,105 and references therein.

In the course of its use for reduction of intraocular pressure, the drug has been reported to cause in some patients an increasing pigmentation and growth of eyelashes. PDR reports that as a side effect its use “may gradually change eyelashes; these changes include increased length, thickness, pigmentation, and number of lashes. These changes in pigmentation and lash growth may be permanent.” U.S. Pat. No. 6,262,105 B1 documented that the use of Latanoprost leads to increased length of lashes, increased numbers of lashes along the normal lash line, increased thickness and luster of lashes, increased auxiliary lash-like terminal hair in transitional areas adjacent to areas of normal lash growth, increased lash-like terminal hairs at the medial and lateral canthal area, increased pigmentation of the lashes, increased numbers, increased length, as well as increased luster, and thickness of fine hair on the skin of the adjacent lid, and finally increased perpendicular angulation of lashes and lash-like terminal hairs. Similar findings on the promotion of eyelash growth using prostaglandin analogs have also been documented in WO 03/009820 and the use of these analogs for the promotion of hair growth on the skin has been suggested. More particularly, WO 03/009820 documented that in clinical studies over up to 12 months, the use of Latanoprost at 0.005% by weight was able to induce changes in color, length, density and thickness of eyelashes in 16.5%, 25.8%, 22.2%, and 17.5% of the subjects tested, respectively.

It occurred to the inventors herein that this reported finding could be employed to advantage by using Latanoprost directly to thicken, lengthen and darken lashes, i.e. in the absence of glaucoma and increased intraocular pressure. The Latanoprost would be used in the form of its solution or preferably incorporated into a conventional mascara or eyelash preparation and applied to the eyelids along the normal lash line.

It is therefore a principle object of the present invention to provide a novel and effective composition and method for using the same for thickening, lengthening and darkening eyelashes for cosmetic purposes.

Another object of the invention is the provision of eyelash growth promotion method which, while effective for its intended purpose, is non-toxic and does not require any undesirable application into the eye itself or coating, adhering, gluing as with false eyelashes onto the eyelids.

### SUMMARY OF THE INVENTION

The present invention relates to the methods and compositions for thickening, lengthening and darkening eyelashes comprising Latanoprost in an ophthalmologically acceptable carrier and medium. Latanoprost, in the form of liquid, gel, emulsion, lotion or other suitable form, is applied topically to the eyelids at the site where the eyelashes are present, along the normal lash line, to achieve the intended purposes.

### DETAILED DESCRIPTION OF THE PREFERRED EMBODIMENT

Latanoprost can be applied in the form of liquid, gel, emulsion, lotion, or other suitable form, such as a mascara formulation to affect the desired functionality of lengthening thickening and darkening eyelashes.

A preferred formulation is in the form of a buffered aqueous solution of Latanoprost having a pH of about 6.7, with each ml of solution containing about 50 μg of Latanoprost (0.005% by weight). Benzalkonium chloride in an amount between 0.001% and 1.0 % by weight, more preferably at about 0.02 % by weight, can be used as a preservative. Other suitable preservatives can also be used, such as thimerosal, chlorobutanol, methyl paraben, propyl paraben, phenylethyl alcohol, edentate disodium, sorbic acid, ONAMER M® and others that are known to those skilled in the art the conventional inactive ingredients such as sodium chloride, sodium dihydrogen phosphate monohydrate, disodium hydrogen phosphate and water can be present.

Latanoprost has limited solubility in water, and therefore an appropriate co-solvent can be added to promote solubility, if desired. Such co-solvents include: Polysorbate 20, 60 and 80; Pluronic F-68, F-84 and P-103; Tyloxapol®; Cremophor® EL; sodium dodecyl sulfate; glycerol; PEG 400; propylene glycol; cyclodextrins; or other agents known to those skilled in the art. Such co-solvent, if desired, can be added in a concentration between about 0.01% and about 2% by weight.

In addition, viscosity-building agents including, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, can be added if desired. The solution can be applied with a dropper or with a brush to the eyelids at the site where the eyelashes are present. If the product is formulated as a mascara or eyeliner, the Latanoprost can be incorporated into the conventional formulations and applied in the usual manner.

In addition, other active agents can be incorporated into the compositions herein in safe and effective amounts. Active ingredients which may be used include antimicrobial, antifungals, antioxidants, pH adjusters, fragrance components and the like.

The applicants' invention is directed to the use of Latanoprost in the form of its aqueous solution, as a gel, cream, emulsion, and the like or in the form of a mascara or eyeliner for increasing length, thickness and pigmentation of eyelashes.

The compositions which are contemplated for utilization in the present invention also contain a solid, semi-solid, or liquid cosmetically or pharmaceutically acceptable vehicle to act as a diluent, dispersant or carrier for the active components in the composition. As used herein, “pharmaceutically-acceptable” means that ingredients which the term describes are suitable for use in humans without undue toxicity, incompatibility, instability, irritation, allergic response, and the like. As used herein, “cosmetically acceptable” means that ingredients which the term describes are suitable for use in contact with the skin without undue toxicity, incompatibility, instability, irritation, allergic response and the like. The cosmetically or pharmaceutically acceptable vehicles comprise from about 0.1% to about 99.999%, preferably from about 25% to about 99.99%, more preferably from about 50% to about 99.99%, even more preferably from about 75% to 99.9%, most preferably from about 85% to about 99.9% by weight of the composition.

Acceptable vehicles include, for example, water, lipophilic or hydrophilic emollients/humectants, surfactants, lubricants, solvents, co-solvents, buffer systems, and preservatives.

Hydrophilic or lipophilic emollients and/or humectants can be incorporated into the compositions herein as the vehicle at levels ranging from about 0.5% to about 85%, preferably from about 5% to about 50%, more preferably from about 10% to about 30% by weight of the composition. Suitable emollients and humectants are listed in CTFA Cosmetic Ingredient Handbook, Second Edition, 1992, pp. 572-575, which is herein incorporated by reference. Suitable emollients/humectants include esters, fatty acids and alcohols, polyols, hydrocarbons, silicones, waxes, triglycerides, cationic and nonionic polymers and mixtures thereof.

Surfactants can be desirably utilized as the vehicle in the compositions herein. Surfactants, if used, are typically employed at levels ranging from about 0.1% to about 30%, preferably from about 1% to about 15%, more preferably from about 0.1% to about 10% by weight of the composition. Suitable surfactants for use herein include cationic, nonionic, anionic, amphoteric and combinations thereof.

Other ingredients which can be employed in the compositions of the present invention are thickeners and binders. A thickener or binder will usually be present in amounts anywhere from 0.01% to 20% by weight, preferably from about 0.1% to about 10%, more preferably from about 0.1% to about 5% by weight of the composition. Suitable thickeners include cross-linked polyacrylate materials available under the trademark Carbopol. Gums may be employed such as xanthan, carrageenan, gelatin, karaya, pectin and locust bean gum. Under certain circumstances the thickening function may be accomplished by a material also serving as a silicone or emollient. For instance, silicone gums in excess of 10 centistokes and esters such as glycerol stearate have dual functionality.

Preferred binders include, but are not limited to methycellulose, sodium carboxymethycellulose, hydroxypropylmethylcellulose, carbomer, polyvinylpyrrolidone, acacia, guar gum, xanthan gum and tragacanth. Particularly preferred are methycellulose, carbomer, xanthan gum, guar gum, polyvinylpyrrolidone and sodium carboxymethycellulose.

The preparations can be applied topically directly onto the normal lash line until the desired effects have been realized or for as long as the individual desired to realize the sought for effects.

The following examples are given for illustrating the invention.

### EXAMPLE 1

The following formulation was prepared in accordance with procedures known to those skilled in the art.

| Ingredient | Amount, weight percent (wt %) |
|---|---|
| Latanoprost | 0.005 |
| Monobasic sodium phosphate | 0.05 |
| Dibasic sodium phosphate (anhydrous) | 0.15 |
| Benzalkonium chloride | 0.02 |
| Sodium chloride | 0.75 |
| HCl and/or NaOH | pH 6.7 |
| Disodium EDTA | 0.05 |
| Purified water | q.s. to 100% |

### EXAMPLE 2

| Ingredient | Parts by weight | |
|---|---|---|
| Latanoprost | 0.005 | wt %. |
| Titanium dioxide | 2.0 | |
| Sodium polyphosphate | 0.05 | |
| Purified water | 35.95 | |
| Polyoxyethylene sorbitan monooleate | 0.5 | |
| Glycerin | 3.0 | |
| Sodium carboxymethyl cellulose | 1.0 | |
| Bentonite | 0.5 | |
| Polymer emulsion | 30 | |
| (Butyl acrylate/2-ethylhexyl acrylate/methyl | | |
| methacrylate = 40/30/30, solid | | |
| content 50% by weight) | | |
| Benzalkonium chloride | 0.02 | wt % |

Although the invention has been described with reference to Latanoprost, in place thereof or in combination there may be used other Minoxidil type compounds, Travoprost, finasteride, finasteride type compounds (dihydrotestosterone blockers) and retinoic acid related compounds.

1. A cosmetic composition for topical use for darkening the color of eyelashes and increasing their length and thickness comprising Latanoprost 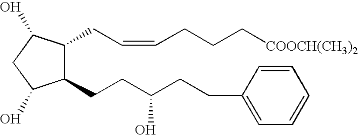
in an opthalmologically acceptable carrier.

2. A cosmetic composition for topical use according to claim 1 in the form of a solution, a thickened liquid, a gel, an emulsion or lotion comprising Latanoprost.

3. A composition for topical use according to claim 1 in the form of its solution.

4. A composition for topical use according to claim 1 in the form a liquid mascara.

5. A method for increasing the thickness, length and for darkening eyelashes which comprises applying to the eyelids in the immediate vicinity of the normal lash line a composition according to claim 1.
